(19) United States
(12) Patent Application Publication
(10) Pub. No.: US 2023/0310098 A1
(43) Pub. Date: Oct. 5, 2023

(54) SURGERY ASSISTANCE DEVICE

(71) Applicant: RIVERFIELD INC., TOKYO (JP)

(72) Inventors: Koh-hei SONODA, Fukuoka (JP); Shintaro NAKAO, Fukuoka (JP); Kataro TADANO, Tokyo (JP); Atsushi MORIKAWA, Tokyo (JP)

(73) Assignee: RIVERFIELD INC., TOKYO (JP)

(21) Appl. No.: 18/306,410

(22) Filed: Apr. 25, 2023

Related U.S. Application Data
(63) Continuation of application No. PCT/JP2020/040230, filed on Oct. 27, 2020.

(51) Int. Cl. A61B 34/30; A61B 34/20; A61F 9/007

(52) U.S. Cl. CPC A61B 34/30; A61B 34/20; A61F 9/007; A61B 2034/301; A61B 2034/305; A61B 2034/2059

(57) ABSTRACT

A surgery assistance device includes an arm that holds an endoscope and adjusts a position of the endoscope, a measuring device having a fixed position relative to the endoscope, the measuring device measuring a distance to a surgery target region of a subject, and a control device configured to obtain a positional relationship between the surgery target region of the subject and a distal end portion of the endoscope based on information from the measuring device, generate a map image showing the position of the endoscope on a three-dimensional model of the surgery target region by using the positional relationship, and transmit display information for displaying the map image.

### CROSS REFERENCE TO RELATED APPLICATIONS

This application is a continuation application of International Patent Application No. PCT/JP2020/040230 filed on Oct. 27, 2020, the contents of which being incorporated by reference herein in its entirety.

### BACKGROUND

The present disclosure relates to a technical field of a surgery assistance device having a function of holding an endoscope.

Vitreous body surgery may restore a retina to a normal state by sucking and removing a vitreous body within an eyeball, for example, for treatment of maculopathy, retinal detachment, or the like.

In related art vitreous body surgery, an operator, such as a surgeon, observes the inside of the eyeball through the pupil of a subject, i.e., a patient, by a surgical microscope or the like. There is a limit to a range in which the inside of the eyeball can be observed through the pupil. In order to bring the part that cannot be observed into a viewable range, the eyeball needs to be pressed from the outside. Such pressing may cause a pain during the surgery or inflammation after the surgery.

A related art method of using an endoscope for vitreous body surgery may be used in which an endoscope is inserted into the eyeball of the subject, and a video of the inside of the eyeball is displayed by display means such as a monitor. The operator can easily observe a part normally unable to be viewed from the pupil by moving the endoscope within the eyeball.

### SUMMARY

It is an aspect to provide a surgery assisting device having a display to enable an operator to recognize a positional relation between the eyeball and the endoscope.

According to an aspect of one or more embodiments, there is provided a surgery assistance device comprising an arm having a holder for holding an endoscope, and configured to adjust a position of the endoscope in a state in which the holder holds the endoscope; a measuring device having a fixed relative positional relationship to the endoscope held by the holder, and used to measure a distance to a surgery target region of a subject; and a control device configured to obtain a positional relationship between the surgery target region of the subject and a distal end portion of the endoscope based on information from the measuring device, to generate a map image indicating the position of the endoscope on a three-dimensional model of the surgery target region by using the positional relationship, and to perform display control of the map image.

According to another aspect of one or more embodiments, there is provided a surgery assistance device comprising an arm configured to hold an endoscope and to adjust a position of the endoscope; a measuring device having a fixed position relative to the endoscope, the measuring device measuring a distance to a surgery target region of a subject; and a control device configured to obtain a positional relationship between the surgery target region of the subject and a distal end portion of the endoscope based on information from the measuring device, generate a map image showing the position of the endoscope on a three-dimensional model of the surgery target region by using the positional relationship, and transmit display information for displaying the map image.

According to yet another aspect of one or more embodiments, there is provided a surgery assistance device comprising an arm configured to hold an endoscope and to adjust a position of the endoscope; a measuring device having a fixed position relative to the endoscope, the measuring device including an irradiating device that irradiates a light spot onto an eyeball of a subject and an imaging device that captures an image of the eyeball; and a control device configured to obtain a positional relationship between the eyeball of the subject and a distal end of the endoscope based on based on the image of the light spot that is included in the captured image and the fixed positon of the measuring device, generate a map image showing the position of the endoscope on a three-dimensional model of the eyeball by using the positional relationship, generate a presenting image including information regarding an insertion length of the endoscope in the eyeball and information regarding a distance from the distal end of the endoscope to a retina of the eyeball, and transmits display information for displaying the map image, the presenting image, and an endoscope image captured by the endoscope.

### BRIEF DESCRIPTION OF DRAWINGS

The above and/or other aspects will become apparent and more readily appreciated from the following description of exemplary embodiments, taken in conjunction with the accompanying drawings in which:

FIG. 1 is a diagram schematically illustrating an example of a configuration of a surgery system, according to some embodiments;

FIG. 2 is a diagram schematically illustrating a sectional configuration of an eyeball of a subject, according to some embodiments;

FIG. 3 is a diagram schematically illustrating an example of a configuration of a surgery assistance device, according to some embodiments;

FIG. 4 is a diagram schematically illustrating an example of a configuration of an arm distal end section of an endoscope holding device, according to some embodiments;

FIG. 5 is a diagram illustrating an example of a display image displayed on a monitor, according to some embodiments;

FIG. 6 is a block diagram illustrating an example of a configuration of the surgery assistance device, according to some embodiments;

FIG. 7 is a flowchart illustrating an example of processing performed by a computation and control device of the surgery assistance device, according to some embodiments; and

FIGS. 8A-8C are diagrams illustrating an outline of a procedure for generating an ocular map image, according to some embodiments.

### DETAILED DESCRIPTION

As described above, in related art vitreous body surgery, a method of using an endoscope for vitreous body surgery may be used in which an endoscope is inserted into the eyeball of the subject, and video of the inside of the eyeball is displayed by display means such as a monitor. The operator can easily observe a part normally unable to be viewed from the pupil by moving the endoscope within the eyeball.

Thus, the vitreous body surgery using such an endoscope obviates a need for pressing the eyeball in observing the inside of the eyeball. A burden on the eyeball of the subject can therefore be reduced.

The vitreous body surgery is performed by inserting a treatment instrument such as a vitreous body cutter, a forceps, or an injector of a perfusate or the like into an eyeball. However, in performing the surgery, the position of an endoscope inserted in the eyeball is not accurately known. Therefore, smooth proceeding of the surgery may be hindered, for example, when the treatment instrument comes into contact with the endoscope during the surgery.

In addition, in inserting the endoscope into the eyeball, when the position of the endoscope in the eyeball cannot be recognized, the endoscope may come into contact with the retina, and damage the retina.

It is accordingly an aspect to provide a surgery assisting device having a display to enable an operator to recognize a positional relation between the eyeball and the endoscope.

A surgery assistance device according to some embodiments includes an arm having a holder for holding an endoscope, and configured to adjust a position of the endoscope in a state in which the holder holds the endoscope, a measuring device having fixed relative positional relation to the endoscope held by the holder, and used to measure a distance to a surgery target region of a subject, a position determining section configured to obtain positional relation between the surgery target region of the subject and the endoscope distal end portion on a basis of information from the measuring device, an image generating section configured to generate a map image indicating the position of the endoscope on a three-dimensional model of the surgery target region by using a determination result of the position determining section, and a display control section configured to perform display control of the map image.

In some embodiments, the surgery target region refers to a region of the subject to be operated by an operator. In some embodiments, the surgery target region may be, for example, the eyeball of the subject in vitreous body surgery.

In some embodiments, the map image may be displayed on a monitor. By checking the monitor, the operator can, for example, recognize the position of the endoscope inserted in the eyeball of the subject.

In some embodiments, the surgery target region may be an eyeball, the measuring device may include an imaging device and an irradiating device, and the position determining section may determine a positional relation between the eyeball of the subject and the endoscope distal end portion on a basis of a captured image of a light spot of light from the irradiating device, the light spot appearing on the eyeball of the subject, the captured image being captured by the imaging device.

For example, in some embodiments, a distance from the imaging device to the eyeball can be calculated on the basis of the captured image. The positional relation between the eyeball and the endoscope distal end can be determined on the basis of the distance.

In some embodiments, the image generating section generates a presenting image including information regarding an insertion length of the endoscope in the eyeball and information regarding a distance from the endoscope distal end portion to a retina of the subject.

In some embodiments, the numerical value of the insertion length of the endoscope in the eyeball and the numerical value of the distance to the retina of the subject, for example, may be displayed on the monitor.

In some embodiments, the display control section may display the map image and a captured image captured by the endoscope (endoscope captured image) within a same screen.

Because the map image and the endoscope captured image are displayed within the same screen, when the operator performs the surgery while checking the captured image of the inside of the eyeball, the operator can easily recognize the positional relation of the endoscope in the eyeball at the same time.

In some embodiments, the image generating section may update the map image according to a shift in the position of the endoscope.

Consequently, the map image illustrating the most recent positional relation between the eyeball and the endoscope may be displayed on the monitor.

According to various embodiments, the operator can perform surgery while recognizing the position of the endoscope inserted in the surgery target region of the subject.

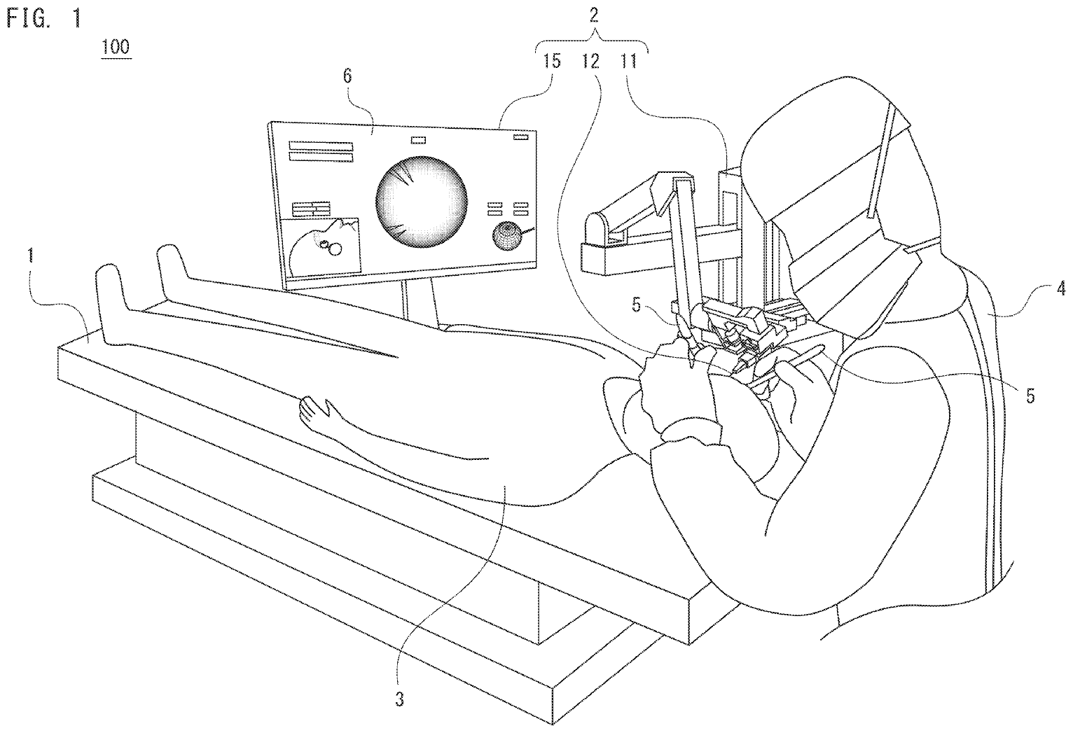

Various embodiments will be described with reference to FIGS. 1 to 8. The drawings extract and illustrate principal parts and peripheral configurations thereof recognized to be necessary for description. In addition, the drawings are schematic, and the dimensions, ratios, and the like of respective structures included in the drawings are mere examples. Hence, various changes can be made according to design or the like without departing from technical ideas of the present invention. In addition, configurations described once may be subsequently identified by the same reference numerals, and description thereof may be omitted.

The various embodiments will hereinafter be described in the following order.
<1. Configuration of Surgery System>
<2. Functional Configuration of Computation and Control Device>
<3. Example of Processing>

<1. Configuration of Surgery System>

A configuration of a surgery system 100 in ocular surgery will be described.

FIG. 1 schematically illustrates an example of the configuration of the surgery system 100, according to some embodiments.

The surgery system 100 includes an operating table 1 and a surgery assistance device 2.

The operating table 1 and the surgery assistance device 2 are installed in an operating room.

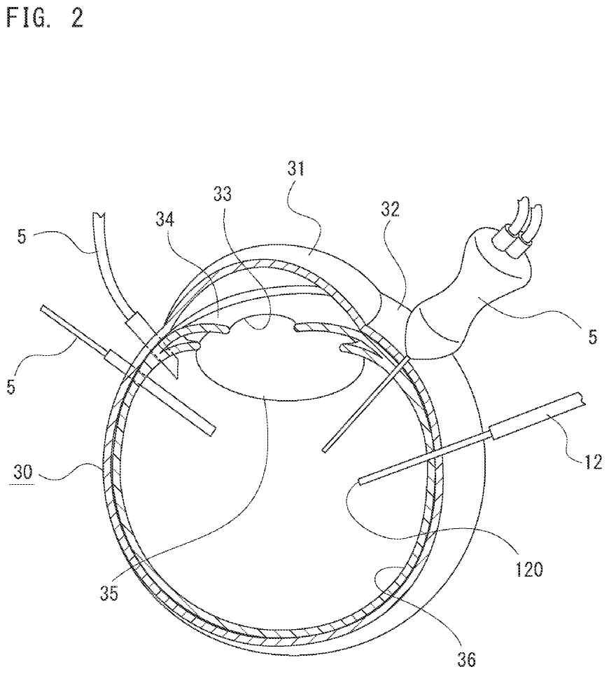

A subject (patient) 3 is laid down on his or her back on the operating table 1. An operator (surgeon) 4 is positioned on the head side of the subject 3, and performs surgery within an eyeball 30 (see FIG. 2) of the subject 3 by using various kinds of treatment instruments 5. Used as the treatment instruments 5 are, for example, a vitreous body cutter, forceps, an injector of a perfusate or the like, and the like.

FIG. 2 schematically illustrates a sectional configuration of the eyeball 30, according to some embodiments. The surface of the eyeball 30 is covered by a cornea 31 and a conjunctiva 32. An iris 34 in which a pupil 33 is formed is present in the rear of the cornea 31. A crystalline lens 35 is present in the rear of the iris 34. In addition, a retina 36 is present on the whole surface of an ocular fundus within the eyeball 30.

The operator 4, for example, inserts the treatment instruments 5 through the conjunctiva 32, and performs surgery within the eyeball 30.

The surgery assistance device 2 assists in the surgery on the eyeball 30 by the operator 4.

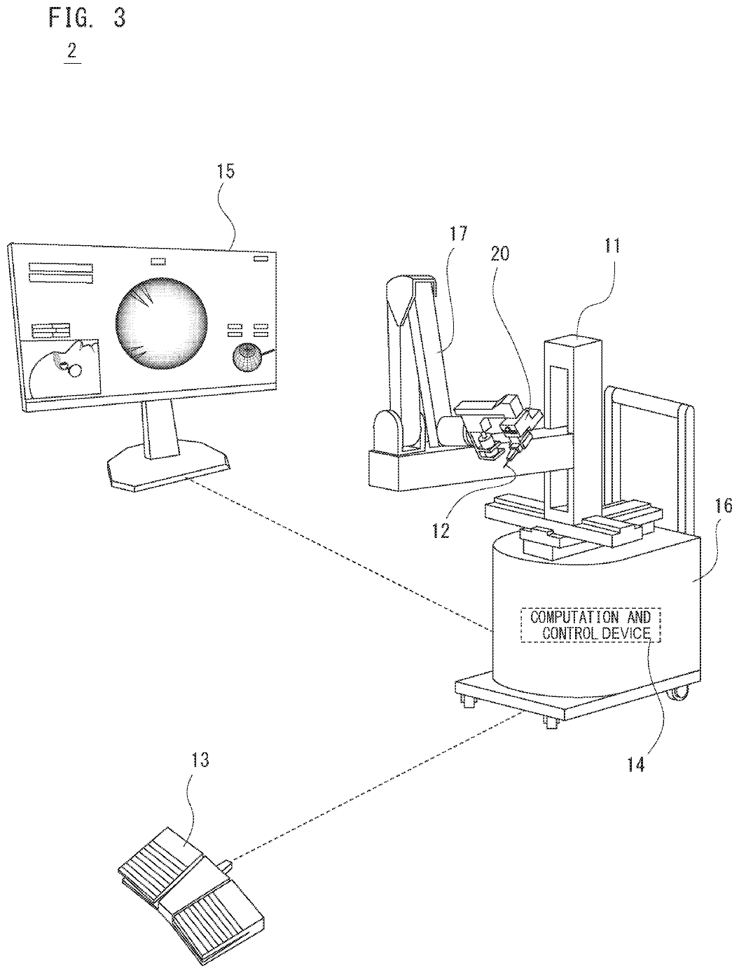

FIG. 3 schematically illustrates an example of a configuration of the surgery assistance device 2, according to some embodiments.

The surgery assistance device 2 includes an endoscope holding device 11, an endoscope 12, an operating device 13, a computation and control device 14, and a monitor 15.

The endoscope holding device 11 includes a base 16 and an arm 17.

The base 16 is mounted on the floor of the operating room or the like. The arm 17 is attached to the base 16. The arm 17 is pivotally supported by the base 16 in a rotatable manner.

The arm 17 includes one or a plurality of joint sections and rotary sections, and is formed as a mechanism that can move an arm distal end section 20 to a given position.

A configuration of the arm distal end section 20 will be described in the following.

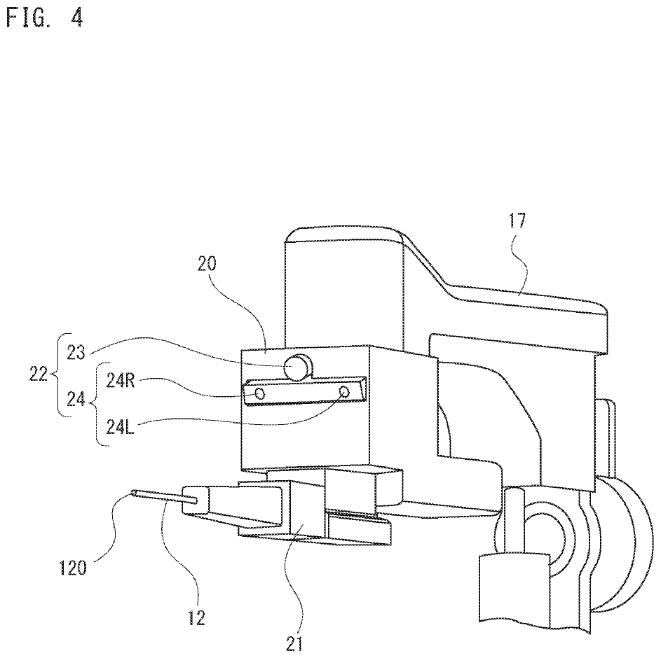

FIG. 4 schematically illustrates an example of the configuration of the arm distal end section 20, according to some embodiments.

The arm distal end section 20 includes a holder 21 for holding the endoscope 12 and a measuring device 22 used to measure a distance to the cornea 31 of the subject 3.

The holder 21 is formed as a mechanism that allows the endoscope 12 to be attached to and detached from the holder 21. The endoscope 12 is fixed to the holder 21 by fitting the endoscope 12 into the holder 21. The endoscope 12 can be freely moved to a given position by operating the arm 17 in a state in which the endoscope 12 is fixed to the holder 21.

When the holder 21 holds the endoscope 12 inserted in the eyeball 30 of the subject 3, the operator 4 does not need to hold the endoscope 12 with a hand. Hence, the operator 4 can perform surgery on the eyeball 30 with both hands.

The measuring device 22 includes an irradiating device 23 and an imaging device 24.

The irradiating device 23 includes an LED (Light Emitting Diode), for example. The irradiating device 23 outputs light that irradiates the eyeball 30 of the subject 3.

The imaging device 24 includes imaging devices 24L and 24R to be able to perform distance measurement by what is called a stereo method. The imaging devices 24L and 24R are, for example, arranged at a predetermined interval from each other in the vicinity of an upper portion of the holder 21. Optical axes of the imaging devices 24L and 24R are parallel with each other, and respective focal lengths of the imaging devices 24L and 24R are the same value. In addition, frame periods thereof are in synchronism with each other, and frame rates thereof coincide with each other.

Captured image signals obtained by respective imaging elements of the imaging devices 24L and 24R are each subjected to A/D (Analog/Digital) conversion, and are thereby converted into digital image signals (captured image data) indicating luminance values based on a predetermined gray scale in pixel units.

Distances from the imaging devices 24L and 24R to the cornea 31 of the subject 3 can be measured on the basis of captured image signals from the respective imaging elements of the imaging devices 24L and 24R, the captured image signals being obtained in a state in which the irradiating device 23 irradiates the eyeball 30. Details of a method of measuring the distances to the cornea 31 of the subject 3 and a method of utilizing the measured distances will be described later.

In the measuring device 22, relative positional relation between the irradiating device 23 and the imaging devices 24L and 24R is fixed. In addition, relative positional relation between the imaging devices 24L and 24R and the above-described holder 21 is fixed. Hence, relative positional relation of the irradiating device 23 and the imaging devices 24L and 24R to the endoscope 12 is fixed by fixing the endoscope 12 to the holder 21.

Returning to FIG. 3, the endoscope 12 of the surgery assistance device 2 is inserted into the eyeball 30 in a state in which the endoscope 12 is fixed to the holder 21 (see FIG. 2). A state within the eyeball 30 is obtained by the inserted endoscope 12. Captured image signals obtained by the imaging element of the endoscope 12 are each subjected to processing, such as A/D conversion, and are thereby converted into digital image signals (captured image data) indicating luminance values based on a predetermined gray scale in pixel units.

A captured image based on the captured image data from the endoscope 12 is displayed on the liquid crystal display of the monitor 15.

The operating device 13 comprehensively represents operating equipment used to perform an operation of the arm 17, a rotational operation of the captured image, which is displayed on the monitor 15, based on imaging by the endoscope 12, and the like. In some embodiments, the operating device 13 may be a controller. In some embodiments, the operating device 13 may be a foot pedal, or in some embodiments may be a manually operated remote operating device (remote controller) or the like. FIG. 3 illustrates a foot pedal as an example. However, as described above, the operating device 13 is not limited to this.

The computation and control device 14 performs various kinds of processing, such as control of operation of the arm 17, processing of generating various kinds of images to be displayed on the monitor 15, and processing of controlling display on the monitor 15.

The computation and control device 14, for example, includes a microcomputer including a CPU (Central Processing Unit), a ROM (Read Only Memory), a RAM (Random Access Memory), and the like. The computation and control device 14 may be implemented by one or a plurality of microcomputers or microprocessors, or by hardware control logic.

The computation and control device 14 may be, for example, included in the base 16 of the endoscope holding unit 11. In some embodiments, the computation and control device 14 may be included in another external apparatus.

The monitor 15 displays a display image 6 on the liquid crystal display under display control from the computation and control device 14.

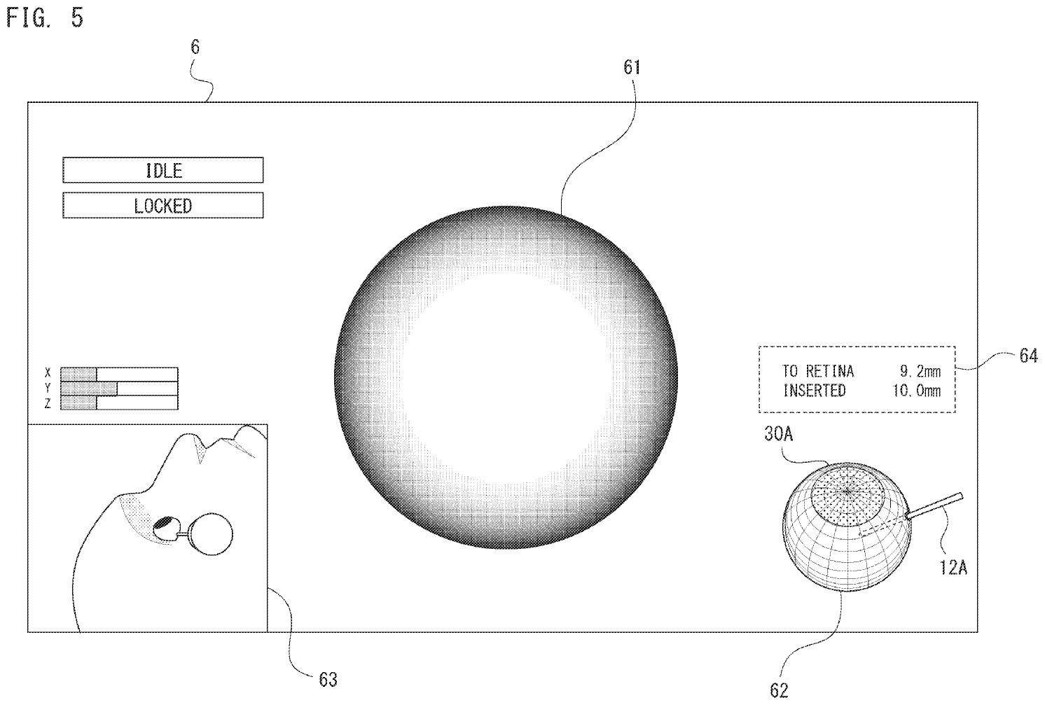

FIG. 5 illustrates an example of the display image 6 displayed on the monitor 15, according to some embodiments.

The display image 6, for example, including an endoscope captured image 61, an ocular map image 62, an endoscope viewpoint map image 63, an insertion length presenting image 64, and the like is displayed on the monitor 15. The display image 6 also includes images related to various kinds of information as required.

The endoscope captured image 61 is the captured image based on the captured image data from the endoscope 12. A state inside the eyeball 30, which is obtained by the endoscope 12, for example, is displayed as the endoscope captured image 61. The endoscope captured image 61 can be rotated by an operation using the operating device 13.

The ocular map image 62 illustrates positional relation between the eyeball 30 and the endoscope 12.

The ocular map image 62 displays the eyeball 30 by a three-dimensional ocular model 30A. In addition, the position of the endoscope 12 with respect to the eyeball 30 is displayed by an endoscope model 12A.

The endoscope viewpoint map image 63 displays a three-dimensional model image of the subject 3 from the viewpoint of the endoscope 12.

The insertion length presenting image 64 displays the numerical value of an insertion length of the endoscope 12 with respect to the eyeball 30 and the numerical value of a distance from an endoscope distal end portion 120 to the retina 36.

The operator 4 performs surgery on the eyeball 30 while checking the display image 6 displayed on the monitor 15.

<2. Functional Configuration of Computation and Control Device>

A functional configuration of the computation and control device 14 in the surgery assistance device 2 will be described.

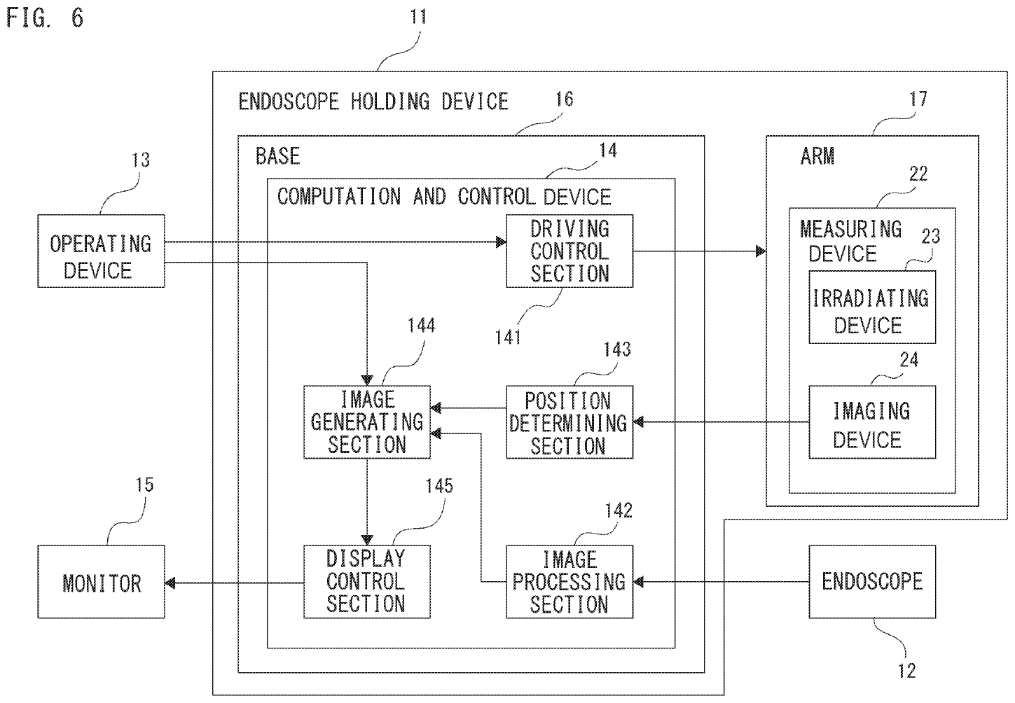

FIG. 6 illustrates, as a block diagram, an example of a configuration of the surgery assistance device 2, according to some embodiments.

The computation and control device 14 includes a driving control section 141, an image processing section 142, a position determining section 143, an image generating section 144, and a display control section 145. As described above, the computation and control device 14 may include a microcomputer including a CPU (Central Processing Unit), a ROM (Read Only Memory), a RAM (Random Access Memory), and the like, that is coded to perform the operations of the driving control section 141, the image processing section 142, the position determining section 143, the image generating section 144, and the display control section 145 described in more detail below.

The driving control section 141 performs operation control on the joint section(s) and the rotary section(s) of the arm 17 of the endoscope holding device 11 on the basis of an operation signal input from the operating device 13, for example. The driving control section 141 can move the position of the endoscope 12 fixed to the holder 21 of the arm distal end section 20 by performing operation control on the arm 17.

In addition, the driving control section 141 performs output control on the irradiating device 23 and imaging control on the imaging device 24.

The image processing section 142 subjects the image signal based on imaging by the endoscope 12 to various kinds of signal processing such as luminance signal processing, color processing, resolution conversion processing, and codec processing. The image processing section 142 outputs the image signal resulting from the various kinds of signal processing to the image generating section 144.

The position determining section 143 obtains distance information from the imaging device 24 to the cornea 31 on the basis of the captured image signals of the eyeball 30 from the respective imaging elements of the imaging devices 24L and 24R, the captured image signals being input from the imaging device 24.

In addition, the position determining section 143 computes relative positional relation between the eyeball 30 and the endoscope distal end portion 120 on the basis of the distance information. Details of a method of computing the relative positional relation will be described later.

The position determining section 143 outputs a determination result (relative positional relation between the eyeball 30 and the endoscope distal end portion 120) to the image generating section 144.

The image generating section 144 generates the display image 6 as illustrated in FIG. 5 by using various kinds of input information from the image processing section 142, the position determining section 143, the operating device 13, and the like. Details of a method for generating various kinds of images constituting the display image 6 will be described later.

The image generating section 144 outputs an image signal of the generated display image 6 to the display control section 145.

The display control section 145 performs control that displays the display image 6 on the monitor 15 on the basis of the image signal input from the image generating section 144.

<3. Example of Processing of Embodiment>

Description will be made of processing performed by the computation and control device 14 of the surgery assistance device 2, according to some embodiments.

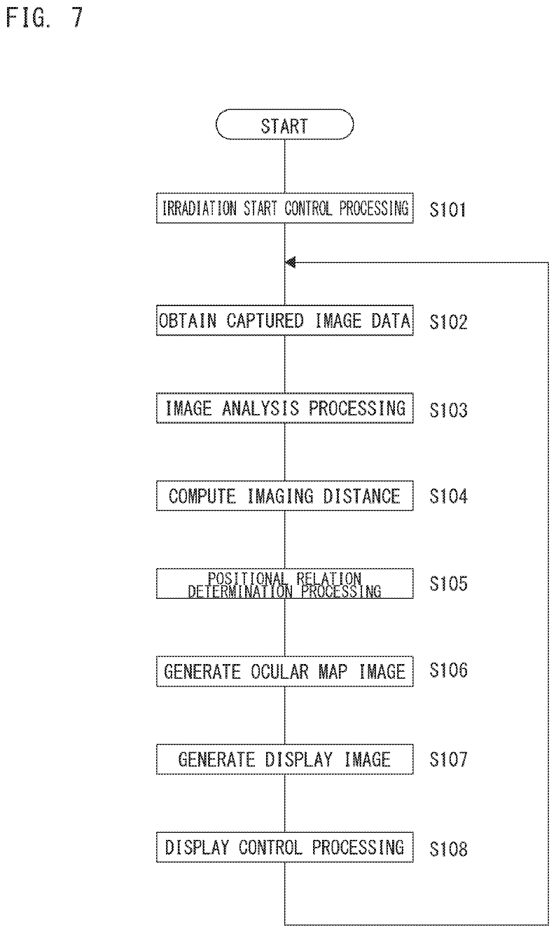

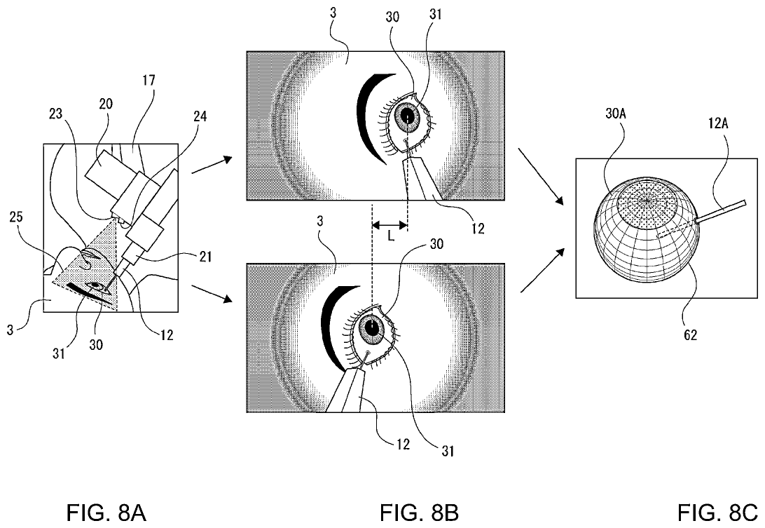

FIG. 7 is a flowchart illustrating an example of processing performed by the computation and control device 14, according to some embodiments. In addition, FIGS. 8A-8C illustrate an outline of a procedure for generating the ocular map image 62, according to some embodiments.

In step S101, the computation and control device 14 performs irradiation start control processing.

In the irradiation start control processing, the computation and control device 14 causes the irradiating device 23 to output light 25 for irradiating the eyeball 30 as illustrated in FIG. 8A. In FIG. 8A, the light 25 output from the irradiating device 23 is schematically indicated by broken lines with shading in between.

Thereafter, the computation and control device 14 repeatedly performs the processing from step S102 to step S109 in timing of each frame of the image.

In step S102, the computation and control device 14 obtains captured image data. For example, the computation and control device 14 stores, in the internal memory, respective pieces of frame image data as the captured image data obtained by imaging the eyeball 30 by the imaging devices 24L and 24R.

In step S103, on the basis of two pieces of captured image data as each frame, the computation and control device 14 performs various kinds of image analysis processing such, for example, as recognition of light spots of the light 25 from the irradiating device 23, which appear on the eyeball 30 or the cornea 31 of the eyeball 30.

In step S104, for the pair of captured image data (stereo images) obtained by the imaging devices 24L and 24R, the computation and control device 14 computes the imaging distance, which is a distance from the imaging device 24 to the cornea 31, by a principle of triangulation from an amount of offset L between the positions of the light spots appearing on the cornea 31, as illustrated in FIG. 8B.

The computation and control device 14 determines a positional relation between the imaging device 24 and the eyeball 30 on the basis of the computed imaging distance.

At this time, data indicating the three-dimensional ocular model 30A as illustrated in FIG. 8C (ocular model data) is used as data on the size of the eyeball 30.

The ocular model data may be, for example, three-dimensional data assuming the eyeball size of an ordinary human. The eyeball size of humans does not differ greatly, though there are slight individual differences. Thus, the standard eyeball size of a human is set in advance as ocular model data.

The computation and control device 14 can compute (determine) the positional relation between the imaging device 24 and the eyeball 30 by using the imaging distance and the ocular model data.

In some embodiments, it is also possible to measure the eyeball size of the eyeball 30 of the subject 3 in advance before the surgery and set the ocular model data by reflecting a result of the measurement.

In the following step S105, the computation and control device 14 determines a positional relation between the endoscope 12 and the eyeball 30 on the basis of the positional relation between the imaging device 24 and the eyeball 30.

The relative positional relation of the endoscope 12 to the imaging device 24 is fixed by fixing the endoscope 12 to the holder 21 of the arm distal end section 20. Therefore, in a state in which the endoscope 12 is fixed to the holder 21, the position of the endoscope 12 is defined naturally according to the position of the imaging device 24.

In some embodiments, a shape of the endoscope 12 up to the endoscope distal end portion 120 in an axial direction of the endoscope 12 fixed to the holder 21 may be known. Therefore, when information regarding the shape of the endoscope 12 is set in advance, the computation and control device 14 can compute the position of the endoscope distal end portion 120 from the defined position of the endoscope 12.

Hence, the computation and control device 14 can compute (determine) the positional relation of the endoscope 12 (endoscope distal end portion 120) to the eyeball 30 on the basis of the positional relation between the eyeball 30 and the imaging device 24.

In the following step S106, the computation and control device 14 generates image data of the ocular map image 62 indicating the determined positional relation between the eyeball 30 and the endoscope 12 (endoscope distal end portion 120) as positional relation between the three-dimensional ocular model 30A and the endoscope model 12A.

In step S107, the computation and control device 14 generates image data of the display image 6.

The computation and control device 14 generates the image data of the display image 6 by synthesizing the endoscope captured image 61, the ocular map image 62, the endoscope viewpoint map image 63, the insertion length presenting image 64, and other necessary images.

Here, description will be made of an example of a method for generating image data of various kinds of images other than the ocular map image 62 described above.

Endoscope Captured Image 61

The computation and control device 14 generates the image data of the endoscope captured image 61 on the basis of the captured image data captured by the endoscope 12.

Endoscope Viewpoint Map Image 63

The computation and control device 14 generates image data of the endoscope viewpoint map image 63 displaying a three-dimensional model image of the subject 3 from the viewpoint of the endoscope 12.

Preset three-dimensional model data of a head portion of a human may be used as the three-dimensional model of the subject 3. A value indicating the angle of the head portion of the subject 3 with respect to the endoscope 12 may be set as head portion angle data in advance on an assumption that the subject 3 is laid down on his or her back on the operating table 1 and an installation angle of the endoscope holding device 11 with respect to the operating table 1 is defined.

The computation and control device 14 generates the three-dimensional model image including the three-dimensional ocular model image on the basis of the three-dimensional model data and the head portion angle data.

Then, the computation and control device 14 generates the image data of the endoscope viewpoint map image 63 by synthesizing the three-dimensional model image and the endoscope model image on the basis of the positional relation between the eyeball 30 and the endoscope 12.

Insertion Length Presenting Image 64

The computation and control device 14 computes information regarding the insertion length of the endoscope 12 in the eyeball 30 and information regarding the distance from the endoscope distal end portion 120 to the retina 36 of the subject 3 from the positional relation of the endoscope 12 (endoscope distal end portion 120) with respect to the ocular model data determined in step S105.

The computation and control device 14 generates the image data of the insertion length presenting image 64 on the basis of the information regarding the insertion length and the information regarding the distance.

The image data of the various kinds of images used to generate the image data of the display image 6 is generated by the method described above.

In the following step S108, the computation and control device 14 performs display control for displaying the display image 6 on the liquid crystal display of the monitor 15. The display image 6 as illustrated in FIG. 5 is thereby displayed within the same screen of the monitor 15.

In some embodiments, the computation and control device 14 may display, on another monitor 15, a part of the endoscope captured image 61, the ocular map image 62, the endoscope viewpoint map image 63, the insertion length presenting image 64, and other necessary images constituting the display image 6.

When the computation and control device 14 completes the processing of step S108, the computation and control device 14 returns the processing to step S102, and thereafter performs similar processing. By repeatedly performing the processing of steps S102 to S108, the computation and control device 14 can update the ocular map image 62 when the position of the endoscope 12 is shifted.

A surgery assistance device 2 according to some embodiments includes an arm 17 having a holder 21 for holding an endoscope 12, and configured to adjust a position of the endoscope 12 in a state in which the holder 21 holds the endoscope 12, a measuring device 22 having fixed relative positional relation to the endoscope 12 held by the holder 21, and used to measure a distance to a surgery target region of a subject 3 (distance to the cornea 31 of the subject 3), a position determining section 143 configured to obtain positional relation between the surgery target region (eyeball 30) of the subject 3 and an endoscope distal end portion 120 on the basis of information from the measuring device 22, an image generating section 144 configured to generate a map image (ocular map image 62) indicating the position of the endoscope 12 (endoscope model 12A) on a three-dimensional model (three-dimensional ocular model 30A) of the surgery target region (eyeball 30) by using a determination result of the position determining section 143, and a display control section 145 configured to perform display control of the map image (ocular map image 62) (see FIG. 6, FIG. 7, and the like).

In some embodiments, the surgery target region may be a region of the subject 3 to be operated by an operator 4. According to some embodiments, the surgery target region may be the eyeball 30 of the subject 3.

Consequently, the ocular map image 62 is displayed on a monitor 15. By checking the monitor 15, the operator 4 can recognize the position of the endoscope 12 inserted in the eyeball 30 of the subject 3.

Hence, the operator 4 can perform surgery while recognizing the position of the endoscope 12 inserted in the eyeball 30 of the subject 3. Hence, according to various embodiments, it is possible to proceed with the surgery on the eyeball 30 smoothly. In addition, the operator 4 can perform the surgery while being aware of the distance of the endoscope distal end portion 120 to the retina 36. The safety of the surgery can therefore be improved.

In the surgery assistance device 2 according to some embodiments, the measuring device 22 includes an irradiating device 23 and an imaging device 24 having fixed relative positional relation to the endoscope 12, and the position determining section 143 determines the positional relation between the eyeball 30 of the subject 3 and the endoscope distal end portion 120 on the basis of a captured image of a light spot of light 25 from the irradiating device 23, the light spot appearing on the cornea 31 of the subject 3, the captured image being captured by the imaging device 24 (see S102 to S105 in FIG. 7, FIG. 8A, FIG. 8B, FIG. 8C, and the like).

A distance from the imaging device 24 to the cornea 31 can be calculated on the basis of the captured image, for example. The positional relation between the eyeball 30 and the endoscope distal end portion 120 can be determined on the basis of the distance.

Hence, it is possible to determine the positional relation between the eyeball 30 and the endoscope distal end portion 120, the positional relation reflecting the position of the subject 3. Consequently, accuracy of determination of the positional relation of the endoscope 12 in the eyeball 30 can be improved, and the operator 4 can recognize the positional relation of the endoscope 12 in the eyeball 30 more accurately.

In the surgery assistance device 2 according to some embodiments, the display control section 145 displays the ocular map image 62 and a captured image captured by the endoscope 12 (endoscope captured image 61) within the same screen (see S108 in FIG. 7 and the like).

Because the ocular map image 62 and the endoscope captured image 61 are displayed within the same screen, when the operator 4 performs the surgery while checking the captured image of the inside of the eyeball 30, the operator 4 easily recognizes the positional relation of the endoscope 12 in the eyeball 30 at the same time. Hence, it is possible to proceed with the surgery on the eyeball 30 more smoothly.

In the surgery assistance device 2 according to some embodiments, the image generating section 144 updates the ocular map image 62 according to a shift in the position of the endoscope 12 (see S108 and S102 in FIG. 7 and the like).

Consequently, the ocular map image 62 indicating the most recent positional relation between the eyeball 30 and the endoscope 12 is displayed on the monitor 15.

Hence, the operator 4 can perform the surgery while recognizing the latest positional relation between the eyeball 30 and the endoscope 12, and can therefore proceed with the surgery on the eyeball 30 more smoothly.

In the surgery assistance device 2 according to some embodiments, the image generating section 144 generates a display image 6 including information regarding an insertion length of the endoscope 12 in the eyeball 30 and information regarding a distance from the endoscope distal end portion 120 to the retina 36 of the subject 3 (see S107 in FIG. 7 and the like).

Consequently, the numerical value of the insertion length of the endoscope 12 in the eyeball 30 and the numerical value of the distance to the retina 36 of the subject 3, for example, are displayed on the monitor 15.

Hence, in addition to visually recognizing the positional relation between the eyeball 30 and the endoscope 12 on the basis of the ocular map image 62, the operator 4 can recognize the positional relation also on the basis of the concrete numerical values.

It is to be noted that, while an example of an intraocular endoscope has been described as an example of the endoscope 12, the endoscope 12 is not limited to the intraocular endoscope. For example, it is possible to apply various endoscopes such as a thoracoscope inserted after an incision between ribs of the subject 3 and a laparoscope inserted after an incision in an abdomen.

In a case of a thoracoscope, for example, the surgery target portion is a chest portion of the subject 3, and the distance from the measuring device 22 to the surgery target portion is a distance from the measuring device 22 to a skin of a rib part of the subject 3. In addition, in a case of a laparoscope, the surgery target portion is various kinds of organs such as a liver of the subject 3, and the distance from the measuring device 22 to the surgery target portion is a distance from the measuring device 22 to a skin of an abdominal region of the subject 3.

Finally, the various embodiments described in the present disclosure are merely illustrative, and the scope of the present disclosure is not limited to the foregoing embodiments. In addition, all of combinations of the configurations described in the embodiment are not necessarily essential to solving the problems discussed above. Further, the effects described in the present disclosure are merely illustrative, and are not limited. Other effects may be produced, or a part of the effects described in the present disclosure may be produced.

1. A surgery assistance device comprising:
an arm having a holder for holding an endoscope, and configured to adjust a position of the endoscope in a state in which the holder holds the endoscope;
a measuring device having a fixed relative positional relationship to the endoscope held by the holder, and used to measure a distance to a surgery target region of a subject; and
a control device configured to obtain a positional relationship between the surgery target region of the subject and a distal end portion of the endoscope based on information from the measuring device, to generate a map image indicating the position of the endoscope on a three-dimensional model of the surgery target region by using the positional relationship, and to perform display control of the map image.

2. The surgery assistance device according to claim 1, wherein:
the surgery target region is an eyeball,
the measuring device includes an imaging device and an irradiating device, and
the control device determines the positional relationship between the eyeball of the subject and distal end portion of the endoscope based on a captured image of a light spot of light from the irradiating device, the light spot appearing on the eyeball of the subject, the captured image being captured by the imaging device.

3. The surgery assistance device according to claim 2, wherein:
the control device generates a presenting image including information regarding an insertion length of the endoscope in the eyeball and information regarding a distance from the distal end portion of the endoscope to a retina of the subject.

4. The surgery assistance device according to claim 3, wherein the control device displays the map image and a captured image captured by the endoscope within a same screen.

5. The surgery assistance device according to claim 4, wherein the control device updates the map image according to a shift in the position of the endoscope.

6. The surgery assistance device according to claim 1, wherein the control device displays the map image and a captured image captured by the endoscope within a same screen.

7. The surgery assistance device according to claim 1, wherein the control device updates the map image according to a shift in the position of the endoscope.

8. A surgery assistance device comprising:
an arm configured to hold an endoscope and to adjust a position of the endoscope;
a measuring device having a fixed position relative to the endoscope, the measuring device measuring a distance to a surgery target region of a subject; and
a control device configured to obtain a positional relationship between the surgery target region of the subject and a distal end portion of the endoscope based on information from the measuring device, generate a map image showing the position of the endoscope on a three-dimensional model of the surgery target region by using the positional relationship, and transmit display information for displaying the map image.

9. The surgery assistance device according to claim 8, wherein the measuring device includes an irradiating device that irradiates a light spot onto the surgery target region and an imaging device that captures an image of the surgery target region, and
wherein the control device determines the positional relationship between the surgery target region and the distal end portion of the endoscope based on the image of the light spot that is included in the captured image.

10. The surgery assistance device according to claim 9, wherein:
the control device generates a presenting image including information regarding an insertion length of the endoscope and information regarding a distance from the distal end portion of the endoscope to a target within the subject.

11. The surgery assistance device according to claim 10, wherein the control device transmits the display information for displaying the map image and an endoscope image captured by the endoscope, within a same screen of a display.

12. The surgery assistance device according to claim 11, wherein the control device updates the map image according to a shift in the position of the endoscope.

13. The surgery assistance device according to claim 8, wherein the control device transmits the display information for displaying the map image and an endoscope image captured by the endoscope, within a same screen of a display.

14. The surgery assistance device according to claim 8, wherein the control device updates the map image according to a shift in the position of the endoscope.

15. A surgery assistance device comprising:
an arm configured to hold an endoscope and to adjust a position of the endoscope;
a measuring device having a fixed position relative to the endoscope, the measuring device including an irradiating device that irradiates a light spot onto an eyeball of a subject and an imaging device that captures an image of the eyeball; and
a control device configured to obtain a positional relationship between the eyeball of the subject and a distal end of the endoscope based on based on the image of the light spot that is included in the captured image and the fixed positon of the measuring device, generate a map image showing the position of the endoscope on a three-dimensional model of the eyeball by using the positional relationship, generate a presenting image including information regarding an insertion length of the endoscope in the eyeball and information regarding a distance from the distal end of the endoscope to a retina of the eyeball, and transmits display information for displaying the map image, the presenting image, and an endoscope image captured by the endoscope.
